(19) United States
(12) Patent Application Publication
(10) Pub. No.: US 2006/0085012 A1
(43) Pub. Date: Apr. 20, 2006

(54) Torquing device delivered over a guidewire to rotate a medical fastener

(75) Inventor: Mark J. Dolan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/952,603

(22) Filed: Sep. 28, 2004

(51) Int. Cl. A61F 2/36

(52) U.S. Cl. 606/104

(57) ABSTRACT

The invention provides an endovascular torquing device for a rotational fastener and a system and method for endovascular delivery of a rotational fastener to a treatment site within a patient. The torquing device comprises a flexible body portion having a guidewire lumen extending longitudinally there through. A coupling portion formed on a distal end of the body portion interfaces with a rotational fastener positioned within a vessel. The system comprises a guide catheter, at least one guidewire, at least one rotational fastener disposed on the guidewire, and a torquing device. The guide catheter is positioned adjacent to a treatment site, and the rotational fastener and guidewire are delivered through the guide catheter to the treatment site. The torquing device, which has a guidewire lumen extending longitudinally through at least a portion of the device, tracks over the guidewire to engage and rotate the fastener.

### TECHNICAL FIELD

This invention relates generally to fasteners for medical purposes. More specifically, the invention relates to a torquing device designed to be delivered over a guidewire to rotate a medical fastener.

### BACKGROUND OF THE INVENTION

Medical screws or fasteners are used for a wide variety of purposes. For example, they affix soft tissues such as ligaments and tendons to bones and bone-like structures, repair fractured bone material, attach pacemaker leads, and repair damaged tissue by attaching one section of tissue to another or by attaching a structure such as a graft over the damaged tissue. Various systems have been used to deliver these fasteners.

U.S. Pat. No. 5,797,918 to McGuire et al. discloses a surgical screwdriver for inserting bone screws in bone tunnels in ligament reconstruction. The screwdriver is used in methods of arthroscopic cruciate ligament reconstruction by introducing a guidewire and then advancing the screwdriver along the guidewire to drive an interference screw mounted on the driver. The screw is held securely and supported by the driver until insertion of the screw is completed, minimizing the chance that the screw will detach from the driver during delivery to the treatment site. Because bone tissue is hard, a substantial amount of tension may then be exerted on the driver to remove it from the screw without simultaneously pulling the screw out of the bone. While effective for treatment of bone, such a method may not be effective when delivering a fastener into soft tissue such as the wall of a blood vessel or hollow organ.

U.S. Pat. No. 5,139,499 to Small et al. discloses a similar screw and driver combination for soft tissue in which a screw is releasably coupled with a driver and, in one embodiment, delivered using a guidewire insertion system. The guidewire has a sharpened distal end that can be pushed into soft tissue at the treatment site. Thus, the risk of losing the screw during the implantation process is minimized by having the guidewire anchored at the point where the screw is to be implanted. However, should the guidewire pull out of the tissue, the screw could be lost at the treatment site. In addition, inserting the guidewire into tissue may cause damage to the tissue.

An implantation device for treating damaged or diseased tissue within the region of the walls of hollow organs is disclosed in U.S. Pat. No. 6,416,522 to Strecker. A pusher is guided through the hollow body of a catheter to deliver securing means such as a nail or a staple. In one embodiment, multiple securing means are received in the hollow body and are implanted one after another by advancing the pusher. This embodiment offers the advantage of simultaneously delivering multiple securing means to a treatment site, but the catheter must be repositioned to deliver each nail or staple to a new location. In addition, the nail or staple may be lost at the treatment site if the catheter is not properly positioned against the vessel wall.

Therefore, it would be desirable to have a device, a system, and a method for endovascular delivery of a medical fastener to a treatment site within a patient that overcome the aforementioned and other disadvantages.

### SUMMARY OF THE INVENTION

One aspect of the present invention is an endovascular torquing device for a rotational fastener. The device comprises a flexible body portion having a guidewire lumen extending longitudinally there through. A coupling portion formed on a distal end of the body portion interfaces with a rotational fastener positioned within a vessel.

Another aspect of the present invention is a system for endovascular delivery of a rotational fastener to a treatment site within a patient. The system comprises a guide catheter, at least one guidewire, at least one rotational fastener disposed on the guidewire, and a torquing device. The torquing device, which has a guidewire lumen extending longitudinally through at least a portion of the device, tracks over the guidewire to engage and rotate the fastener.

Another aspect of the present invention is a method for endovascular delivery of a rotational fastener to a treatment site within a patient. A guide catheter is positioned adjacent to the treatment site. At least one guidewire having a rotational fastener disposed on the guidewire is delivered through the guide catheter to the treatment site. A torquing device is inserted into the guide catheter over the guidewire and delivered to a location adjacent to the rotational fastener. The torquing device is rotated such that it applies a torque to the rotational fastener, thereby rotating the fastener. The torquing device and the guidewire are then withdrawn from the guide catheter.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of an endovascular torquing device for a rotational fastener, in accordance with the present invention, as seen from the side of the device;

FIGS. 2A through 2E are illustrations of embodiments of endovascular torquing devices, in accordance with the present invention, as seen from the distal end of the device;

FIG. 3 is an illustration of one embodiment of a system for endovascular delivery of a rotational fastener to a treatment site within a patient, in accordance with the present invention;

FIG. 4 is an illustration of another embodiment of a system for endovascular delivery of a rotational fastener to a treatment site within a patient, in accordance with the present invention;

FIG. 5 is a flow diagram of one embodiment of a method for endovascular delivery of a rotational fastener to a treatment site within a patient, in accordance with the present invention.

### DETAILED DESCRIPTION OF THE PRESENTLY PREFERRED EMBODIMENTS

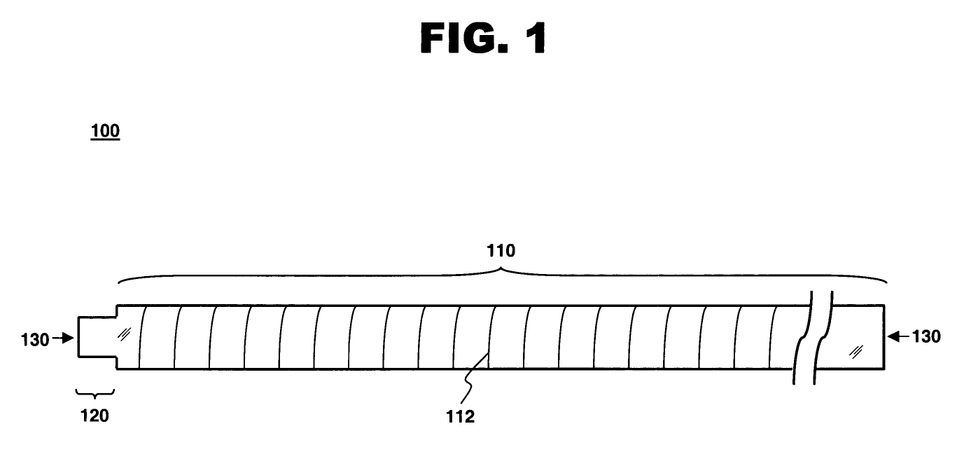

One aspect of the present invention is an endovascular torquing device for a rotational fastener. One embodiment of the device, in accordance with the present invention, is illustrated in FIG. 1 at 100. Device 100 comprises a flexible body portion 110, a coupling portion 120, and a guidewire lumen 130 extending longitudinally through the device. The coupling portion interfaces with a rotational fastener such as a screw or a spiral (not shown) that is positioned within a vessel.

Flexible body portion 110 may be made using any suitable biocompatible material, including, but not limited to, stainless steel, nitinol, polymeric materials, and combinations thereof. In the present embodiment, flexible body portion 110 is a nitinol hypotube. Portion 110 is sized to extend outside the body of a patient when in use; however, in another embodiment, a tube with a guidewire lumen may be inserted into the proximal end of the body portion to extend the torquing device to the length desired.

Body portion 110 includes a spiral cut 112 formed such that when device 100 is rotated, the spiral tightens against itself rather than unwinding. Thus, the spiral cut increases flexibility of the body portion but without limiting transmission of torque to the rotational fastener. One skilled in the art will recognize that while a spiral cut provides a convenient, inexpensive means for increasing flexibility of the body portion, various other structures and methods may be employed to accomplish a similar result.

In the present embodiment, coupling portion 120 is formed from the same hypotube and, therefore, comprises the same material as body portion 110. In another embodiment, the coupling portion may be a separate structure formed using a material that is the same as or different from that used to form the body portion. A separate structure may be attached to the body portion by a method such as adhesive bonding or soldering.

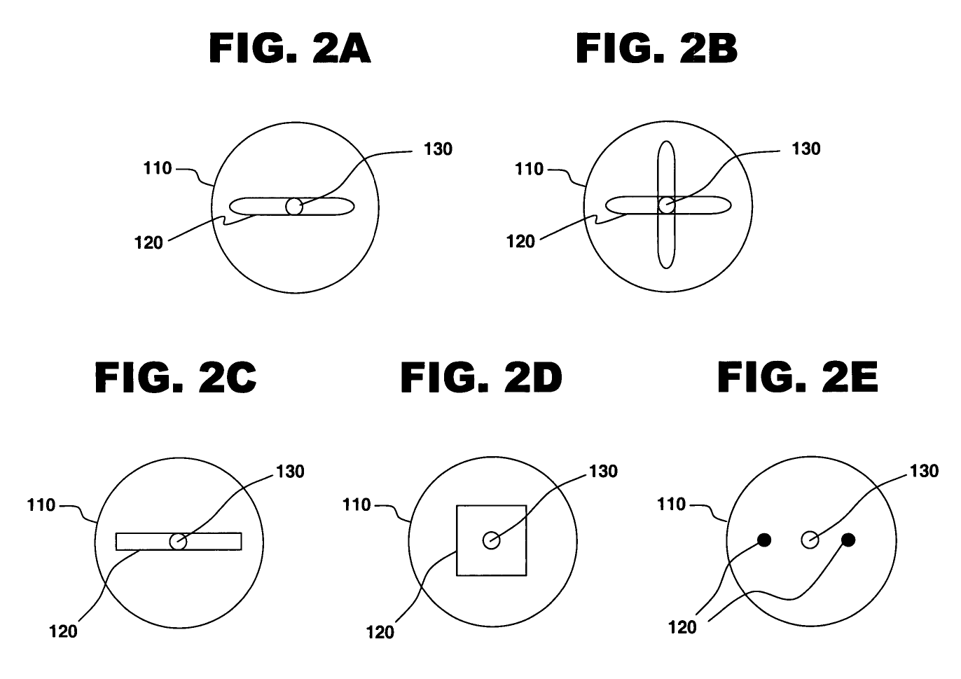

As shown in FIG. 1, coupling portion 120 is an extended coupling segment that tapers and flattens out to a tip that is complementary to a slotted head in a screw or a slotted opening at the end of a spiral. FIGS. 2A through 2E, in which like elements share like reference numbers with FIG. 1, show front views of several exemplary shapes for the coupling portion, FIG. 2A representing a coupling segment that forms a slot driver, FIG. 2B a philips driver, FIG. 2C a Heli-Coil® (Emhart Inc.) driver, FIG. 2D a high torque driver, and FIG. 2E a spanner driver. One skilled in the art will recognize that various other shapes are possible. Each extended coupling segment interfaces with a complementary receptacle in the rotational fastener. Alternatively, the torquing device coupling portion may include a coupling receptacle, while the rotational fastener has a complementary extended coupling segment.

As seen in FIG. 1, guidewire lumen 130 extends longitudinally through both body portion 110 and coupling portion 120 of torquing device 100. Thus, the entire device tracks over a guidewire to interface with a rotational fastener. In another embodiment, the guidewire lumen may extend through only the body portion of the torquing device, with the lumen exiting adjacent to the coupling portion of the device. The coupling portion would thus be positioned adjacent to a guidewire running through the guidewire lumen of the body portion.

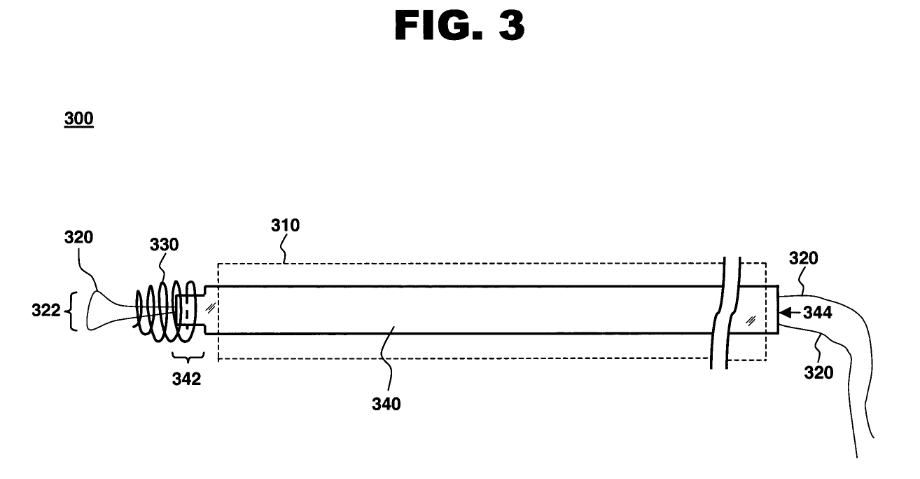

Another aspect of the present invention is a system for endovascular delivery of a rotational fastener to a treatment site within a patient. One embodiment of the system, in accordance with the present invention, is illustrated in FIG. 3 at 300. System 300 comprises a guide catheter 310, a guidewire 320, a rotational fastener 330, and a torquing device 340.

Guide catheter 310 may be any appropriate guide catheter known in the art. A guidewire 320 extends through guide catheter 310. In the present embodiment, the guidewire is roughly doubled over onto itself with the ends of the guidewire adjacent to each other. Guidewire 320 is sized such that the ends of the guidewire extend through guide catheter 310 and outside the patient when rotational fastener 330 is positioned adjacent to a treatment site. A mid-portion 322 of guidewire 320 may be looped about a medical device into or through which the fastener is to be rotated or about a portion of the fastener itself, thus anchoring the guidewire.

Torquing device 340 is shown with guidewire 320 extending through the torquing device guidewire lumen 342, the device having tracked simultaneously over two portions of guidewire 320 to fastener 330. A distal portion of torquing device 340 includes a coupling structure 344 that is complementary to a proximal portion of fastener 330 and is thus able to engage and rotate the fastener. As shown in FIG. 3, coupling structure 344 is an extended coupling segment that tapers and flattens out to a tip that is complementary to a receptacle formed within the spirals of fastener 330. One skilled in the art will recognize that numerous shapes are possible for rotational fastener 330, and, therefore, extended coupling structure 342 may assume a variety of shapes complementary to the fastener. In another embodiment, the torquing device coupling structure may be a coupling receptacle that is complementary to an extended coupling segment included on a fastener.

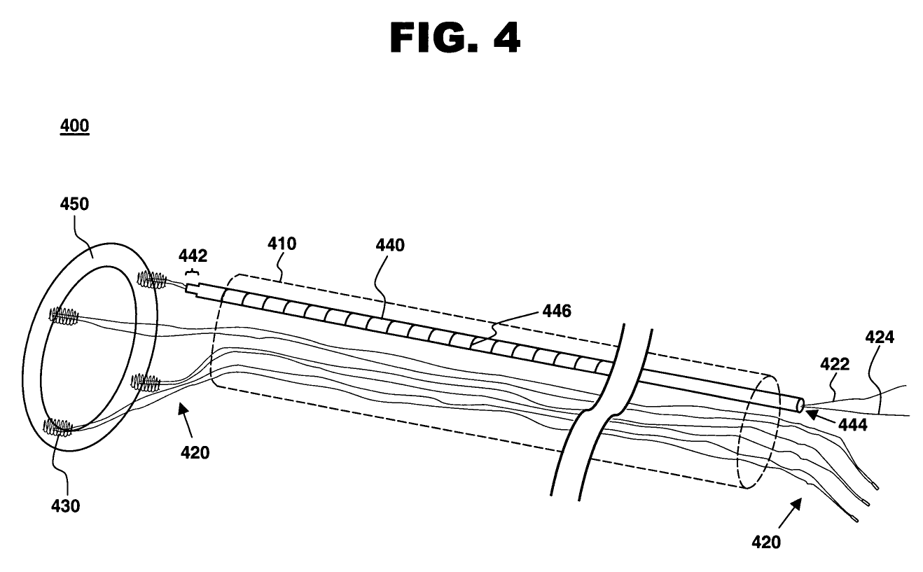

Another embodiment of a system for endovascular delivery of a rotational fastener to a treatment site within a patient, in accordance with the present invention, is illustrated in FIG. 4 at 400. System 400 comprises a guide catheter 410, four guidewires 420, four rotational fasteners 430, a torquing device 440, and a medical device 450. Guide catheter 410 may be any catheter known in the art that is appropriate for delivering a medical device to a treatment site within a vessel. Medical device 450 is sized for delivery through guide catheter 410. The medical device may be folded or otherwise compressed to minimize the delivery profile of the device. In the present embodiment, medical device 450 is a ring such as might be used for mitral valve repair. The four guidewires 420 are removably attached to ring 450 by passing each guidewire through a portion of the ring, thereby looping a mid-portion of the guidewire about the ring. Each guidewire is roughly doubled over onto itself with the ends of the guidewire adjacent to each other and two portions of the guidewire extending away from the medical device. The guidewires are sized such that the ends of the guidewires extend through guide catheter 410 and outside the patient when ring 450 is positioned adjacent to the treatment site.

In the present embodiment, rotational fasteners 430 are shown as spirals. However, it will be apparent to one skilled in the art that the shape and number of fasteners may be varied as needed for a particular medical device. Rotational fasteners 430 are preset into ring 450 before the ring is delivered through guide catheter 310 to the treatment site. Each fastener is positioned over a guidewire such that the two portions of the guidewire extending away from ring 450 pass through the fastener.

In an alternative embodiment, the rotational fasteners may be preset into the ring with the two portions of the guidewire passing on either side of the fastener rather than through the fastener. Thus, the rotational fastener is disposed on the guidewire by having portions of the guidewire running alongside the fastener. In another alternative embodiment, the guidewire may be removably attached to the preset rotational fastener instead of to the medical device, with the guidewire looped about the fastener as described above for the medical device. In yet another alternative embodiment, the rotational fastener may not be preset into the medical device but may be passed over a guidewire attached to the medical device after the device has been delivered through the guide catheter.

A distal portion of torquing device 440 includes a coupling structure 442 that is complementary to a proximal portion of fastener 430. As shown in FIG. 4, coupling structure 442 is an extended coupling segment that tapers and flattens out to a tip that is complementary to a receptacle formed within the spirals of rotational fastener 430. One skilled in the art will recognize that numerous shapes are possible for rotational fastener 430, and, therefore, extended coupling structure 442 may assume a variety of shapes complementary to the fastener. The coupling structure on torquing device 440 may also be a coupling receptacle that is complementary to an extended coupling segment included on a fastener.

Once ring 450 and the four preset fasteners 430 are in place at the treatment site, torquing device 440 is placed over one of the four guidewires, a guidewire lumen 442 extending longitudinally through at least a portion of the torquing device accommodating the guidewire. The device tracks down the guidewire, engages the fastener associated with that guidewire, rotates the fastener into place, and is then withdrawn from the guide catheter for repositioning over another guidewire. This process continues until all four fasteners have been rotated into place. The two ends of each guidewire may be bound one to the other as seen in FIG. 4 to facilitate passing the torquing device over the two guidewire portions simultaneously.

In the present embodiment, torquing device 440 includes a spiral cut 446 that allows the device to bend easily around curves in a vessel. One skilled in the art will recognize that while a spiral cut provides a convenient, inexpensive means for increasing flexibility of the torquing device, various other structures and methods may be employed to accomplish a similar result.

Once the fasteners have been rotated into place, torquing device 440 may be removed and the guidewires may be withdrawn by releasing one end of a guidewire, for example the end labeled 422, and pulling on the other end, end 424, until the guidewire is withdrawn from the vessel. Each individual guidewire may be removed after the torquing device has rotated the associated fastener and been withdrawn from the guidewire. This provides a convenient way of knowing which fasteners have not yet been rotated into place.

In the present embodiment, fasteners 430 are rotated into tissue at the treatment site to secure ring 450 to the treatment site. In an alternative embodiment, rotating the fasteners may secure one medical device to another medical device already present at the treatment site.

In yet another alternative embodiment, a medical device into which one or more fasteners have been preset may be no more than a delivery platform for the fasteners, which pass entirely through the platform upon rotation. For example, such removable fasteners may be used to pin one section of tissue to another or to provide an anchoring device or an eyelet for a wire or other structure. Where the medical device is simply a delivery platform, the device is withdrawn from the treatment site following delivery of the fasteners and may be pulled back through the guide catheter using guidewires that remain in place on the device.

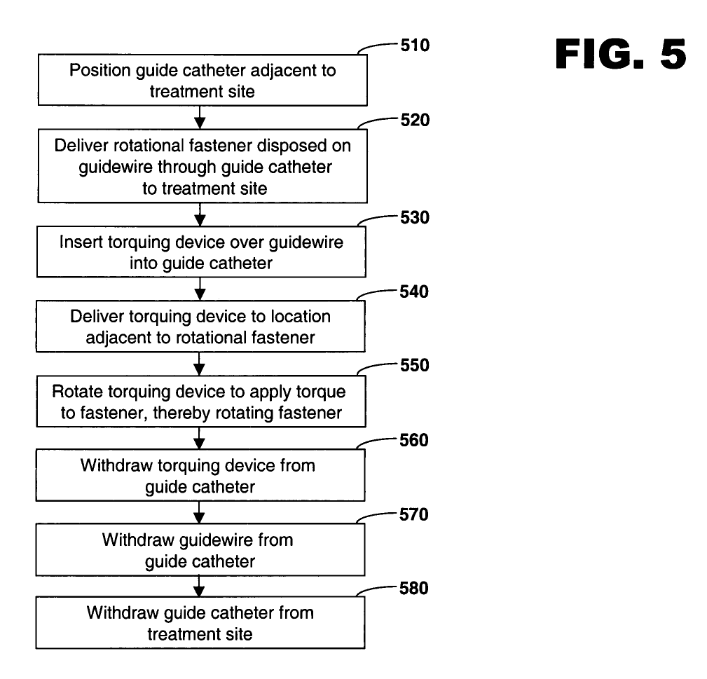

A further aspect of the present invention is a method for endovascular delivery of a rotational fastener to a treatment site within a patient. FIG. 5 shows a flow diagram of one embodiment of the method in accordance with the present invention.

A guide catheter is positioned adjacent to a treatment site (Block 510). This may be accomplished by creating a percutaneous access site in a vessel to be treated or in a vessel that leads to the treatment site. The guide catheter is then introduced through the percutaneous access site and advanced to a position adjacent to the treatment site.

At least one guidewire having a rotational fastener disposed on the guidewire is delivered through the guide catheter to the treatment site (Block 520). As just one example, the guidewire may be removably attached to a medical device by passing a portion of the guidewire through a portion of the medical device, thereby looping the guidewire about the medical device. The ends of the guidewire are adjacent to one another and extend outside the patient when the medical device is positioned adjacent to the treatment site. A fastener is paced over the guidewire such that the two portions of the guidewire extending away from the medical device pass through the fastener. In this example, delivering the fastener disposed on the guidewire comprises delivering the medical device with the attached guidewire and fastener through the guide catheter to the treatment site.

A torquing device is inserted into the guide catheter over the guidewire (Block 530) and is delivered to a location adjacent to the rotational fastener (Block 540). Because the torquing device tracks over the guidewire, it is automatically directed to the rotational fastener, ensuring the torquing device is in proper position to engage the fastener.

The torquing device is rotated such that it applies a torque to the rotational fastener, thereby rotating the fastener (Block 550). This may be accomplished when either a coupling receptacle or an extended coupling segment included on the distal end of the torquing device engages a complementary structure included on a proximal portion of the fastener, allowing the fastener to be rotated by the torquing device.

After rotating the fastener, the torquing device is withdrawn from the guide catheter (Block 560). Where more than one rotational fastener is preset into a medical device, the torquing device is placed over and tracks down a first guidewire, engages the fastener associated with that guidewire, rotates the fastener into place, and is then withdrawn from the guide catheter and repositioned over the next guidewire. This process continues until all of the fasteners have been satisfactorily rotated into place, at which point the torquing device is permanently removed from the guide catheter.

The guidewire is then withdrawn from the guide catheter (Block 570). Where more than one rotational fastener is involved, each guidewire may be removed individually after the torquing device has tracked down that guidewire, rotated the associated fastener, and been withdrawn from the guidewire. This provides a convenient way of knowing which fasteners have not yet been rotated into place. In the present example, removing a guidewire is accomplished by releasing a first end of the guidewire and pulling on a second end of the guidewire until the guidewire is withdrawn from the vessel.

Once all of the fasteners have been rotated into place and the guidewire(s) and torquing device removed, the guide catheter may then be used for performing additional procedures or may be withdrawn from the treatment site and removed from the patient (Block 580).

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. An endovascular torquing device for a rotational fastener, comprising:
a flexible body portion having a guidewire lumen extending longitudinally there through; and
a coupling portion formed on a distal end of the body portion, wherein the coupling portion interfaces with a rotational fastener positioned within a vessel.

2. The device of claim 1 wherein the guidewire lumen extends through the coupling portion.

3. The device of claim 1 wherein the coupling portion includes one of a coupling receptacle or an extended coupling segment complementary to the rotational fastener.

4. The device of claim 1 wherein the flexible body portion has a spiral cut.

5. The device of claim 1 further comprising an extender having a guidewire lumen, wherein the extender is inserted into the proximal end of the body portion to extend the torquing device to the desired length.

6. A system for endovascular delivery of a rotational fastener to a treatment site within a patient, comprising:
a guide catheter;
at least one guidewire;
at least one rotational fastener disposed on the guidewire; and
a torquing device having a guidewire lumen extending longitudinally through at least a portion of the device, wherein the torquing device tracks over the guidewire to engage and rotate the fastener.

7. The system of claim 6 wherein a distal portion of the torquing device includes a coupling structure, and wherein the coupling structure is complementary to a proximal portion of the rotational fastener.

8. The system of claim 6 wherein the coupling structure is one of a coupling receptacle or an extended coupling segment.

9. The system of claim 6 wherein a distal portion of the torquing device has a spiral cut.

10. The system of claim 6 wherein the torquing device tracks over a first and a second portion of the guidewire simultaneously.

11. The system of claim 6 wherein a mid-portion of the guidewire is looped about a portion of the rotational fastener such that the ends of the guidewire are adjacent to one another, the guidewire being sized such that the ends of the guidewire extend outside the patient when the rotational fastener is positioned adjacent to the treatment site.

12. The system of claim 6 further comprising:
a medical device sized for delivery through the guide catheter, wherein the rotational fastener is further disposed on the medical device.

13. The system of claim 12 wherein a mid-portion of the guidewire is looped about a portion of the medical device such that the ends of the guidewire are adjacent to one another, the guidewire being sized such that the ends of the guidewire extend outside the patient when the medical device is positioned adjacent to the treatment site.

14. The system of claim 12 wherein at least one rotational fastener secures the medical device to the treatment site.

15. The system of claim 12 wherein the medical device delivers at least one rotational fastener to the treatment site and is withdrawn from the treatment site following delivery of the at least one fastener.

16. The system of claim 12 wherein the rotational fastener is disposed on the medical device after the medical device has been delivered through the guide catheter.

17. A method for endovascular delivery of a rotational fastener to a treatment site within a patient, comprising:
positioning a guide catheter adjacent to the treatment site;
delivering at least one guidewire having a rotational fastener disposed on the guidewire through the guide catheter to the treatment site;
inserting a torquing device into the guide catheter over the guidewire;
delivering the torquing device to a location adjacent to the rotational fastener;
rotating the torquing device such that it applies a torque to the rotational fastener, thereby rotating the fastener;
withdrawing the torquing device from the guide catheter; and
withdrawing the guidewire from the guide catheter.

18. The method of claim 16 wherein the system further comprises a medical device sized for delivery through the guide catheter, and wherein the guidewire is removably attached to the medical device.

19. The method of claim 18 wherein delivering the guidewire having a rotational fastener disposed thereon through the guide catheter to the treatment site comprises delivering the medical device to the treatment site.

20. The method of claim 18 wherein a mid-portion of the guidewire is looped about one of the medical device or the rotational fastener such that the ends of the guidewire are adjacent to one another, the guidewire being sized such that the ends of the guidewire extend outside the patient when the rotational fastener is positioned adjacent to the treatment site, and wherein withdrawing the guidewire from the guide catheter comprises releasing a first end of the guidewire and pulling on a second end of the guidewire until the guidewire is withdrawn from the vessel.
